(19) United States
(12) Patent Application Publication
(10) Pub. No.: US 2025/0386659 A1
(43) Pub. Date: Dec. 18, 2025

(54) DETECTION DEVICE

(71) Applicant: Japan Display Inc., Tokyo (JP)

(72) Inventor: Genki ASOZU, Tokyo (JP)

(21) Appl. No.: 19/313,450

(22) Filed: Aug. 28, 2025

Related U.S. Application Data
(63) Continuation of application No. PCT/JP2024/005911, filed on Feb. 20, 2024.

(30) Foreign Application Priority Data
Mar. 3, 2023 (JP) 2023-032743

(51) Int. Cl. H10K 39/00; G06F 1/16; H10K 39/38

(52) U.S. Cl. CPC H10K 39/601; G06F 1/163; G06F 1/1635; H10K 39/38

(57) ABSTRACT

According to an aspect, a detection device includes: a substrate; an organic optical sensor in which a first lower electrode, a first lower buffer layer, a first active layer, a first upper buffer layer, a first upper electrode, and a common electrode are stacked in the order as listed, in a detection area of the substrate; an organic photovoltaic device in which a second lower electrode, a second lower buffer layer, a second active layer, a second upper buffer layer, and the common electrode are stacked in the order as listed, in the detection area of the substrate; and a sealing film that covers the organic optical sensor and the organic photovoltaic device. The first lower electrode of the organic optical sensor and the common electrode of the organic photovoltaic device each have a light-transmitting property.

### CROSS-REFERENCE TO RELATED APPLICATION

This application claims the benefit of priority from Japanese Patent Application No. 2023-032743 filed on March 3, 2023 and International Patent Application No. PCT/JP2024/005911 filed on February 20, 2024, the entire contents of which are incorporated herein by reference.

### BACKGROUND

### Technical Field

What is disclosed herein relates to a detection device.

### Description of the Related Art

WO 2016/104517 describes a biosensor that includes at least three types of sensor elements. WO 2016/104517 mentions, as an example of the sensor elements, an optical sensor element that senses visible light and/or fluorescence using a sensing unit. Such an optical sensor includes a plurality of photodiodes (organic photodiodes (OPDs)) each using an organic semiconductor material as an active layer.

A current flows through each of the OPDs used in a detection device when irradiated with light. Therefore, the OPD has a configuration similar to that of a photovoltaic device such as a solar cell. However, even when some of the OPDs of the detection device are used as the photovoltaic devices (solar cells) as they are, power may not be generated satisfactorily.

For the foregoing reasons, there is a need for a detection device that includes an organic optical sensor and an organic photovoltaic device and is capable of detecting light and generating power satisfactorily.

### SUMMARY

According to an aspect, a detection device includes: a substrate; an organic optical sensor in which a first lower electrode, a first lower buffer layer, a first active layer, a first upper buffer layer, a first upper electrode, and a common electrode are stacked in the order as listed, in a detection area of the substrate; an organic photovoltaic device in which a second lower electrode, a second lower buffer layer, a second active layer, a second upper buffer layer, and the common electrode are stacked in the order as listed, in the detection area of the substrate; and a sealing film that covers the organic optical sensor and the organic photovoltaic device. The first lower electrode of the organic optical sensor and the common electrode of the organic photovoltaic device each have a light-transmitting property.

According to an aspect, a detection device includes: a substrate; an organic optical sensor in which a first lower electrode, a first lower buffer layer, a first active layer, a first upper buffer layer, a first upper electrode, and a common electrode are stacked in the order as listed, in a detection area of the substrate; an organic photovoltaic device in which a second lower electrode, a second lower buffer layer, a second active layer, a second upper buffer layer, a second upper electrode, and the common electrode are stacked in the order as listed, in the detection area of the substrate; and a sealing film that covers the organic optical sensor and the organic photovoltaic device. The first upper electrode and the common electrode of the organic optical sensor and the second lower electrode of the organic photovoltaic device each have a light-transmitting property. The second upper electrode of the organic photovoltaic device has no light- transmitting property.

### BRIEF DESCRIPTION OF THE DRAWINGS

FIG. 1 is a schematic view illustrating an exemplary external appearance when a state of a finger accommodated

inside a detection device according to an embodiment is viewed from a lateral side of a housing;

FIG. 2 is a sectional view taken along section II-II' of FIG. 1;

FIG. 3 is a plan view schematically illustrating the detection device according to the embodiment;

FIG. 4 is a plan view schematically illustrating an

arrangement example of optical sensors and solar cells in a detection area;

FIG. 5 is a block diagram illustrating a configuration example of the detection device according to the embodiment;

FIG. 6 is a circuit diagram illustrating the detection device according to the embodiment;

FIG. 7 is a block diagram schematically illustrating a configuration example of the optical sensors, the solar cells, a battery circuit, and light sources;

FIG. 8 is a sectional view taken along section VIII- VIII' of FIG. 4;

FIG. 9 is an enlarged sectional view illustrating one of the optical sensors in FIG. 8;

FIG. 10 is a sectional view taken along section X-X' of FIG. 4;

FIG. 11 is an enlarged sectional view illustrating each of the solar cells in FIG. 8;

FIG. 12 is an explanatory diagram for explaining a relation between detection by the optical sensors of the detection device and operation of the solar cells;

FIG. 13 is a sectional view of a detection device according to a modification of the embodiment; and

FIG. 14 is an enlarged sectional view of the solar cell in FIG. 13.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present

disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### Embodiment

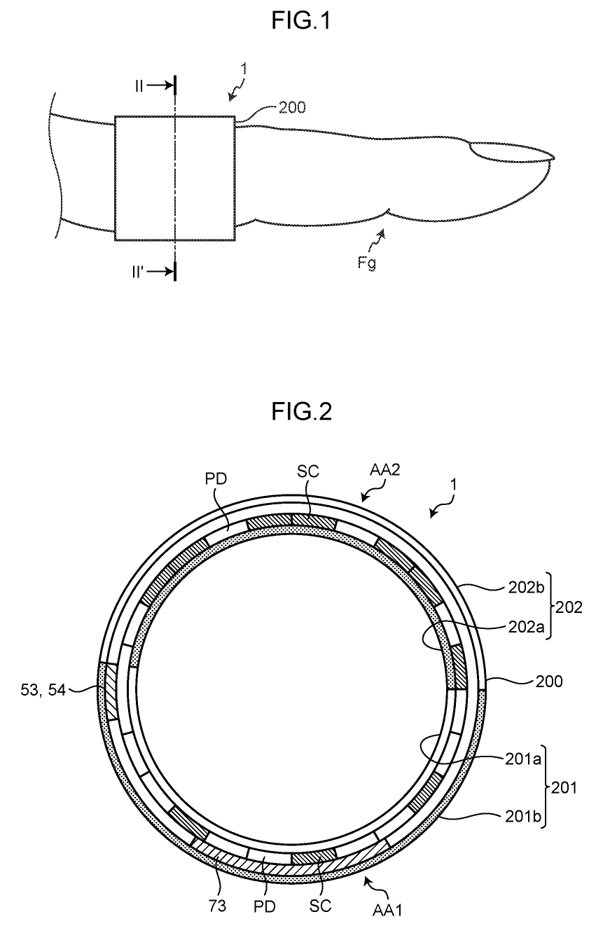

FIG. 1 is a schematic view illustrating an exemplary external appearance when a state of a finger accommodated inside a detection device according to an embodiment is viewed from a lateral side of a housing. FIG. 2 is a sectional view taken along section II-II' of FIG. 1.

A detection device 1 illustrated in FIGS. 1 and 2 is a finger ring-shaped device that can be worn on and removed from a human body and is worn on an object to be detected Fg of the human body. The object to be detected Fg of the embodiment is a digit (finger or the like), and may be any one of a thumb, an index finger, a middle finger, a ring finger, and a little finger. The detection device 1 can detect biometric information on a living body from the object to be detected Fg wearing the detection device 1.

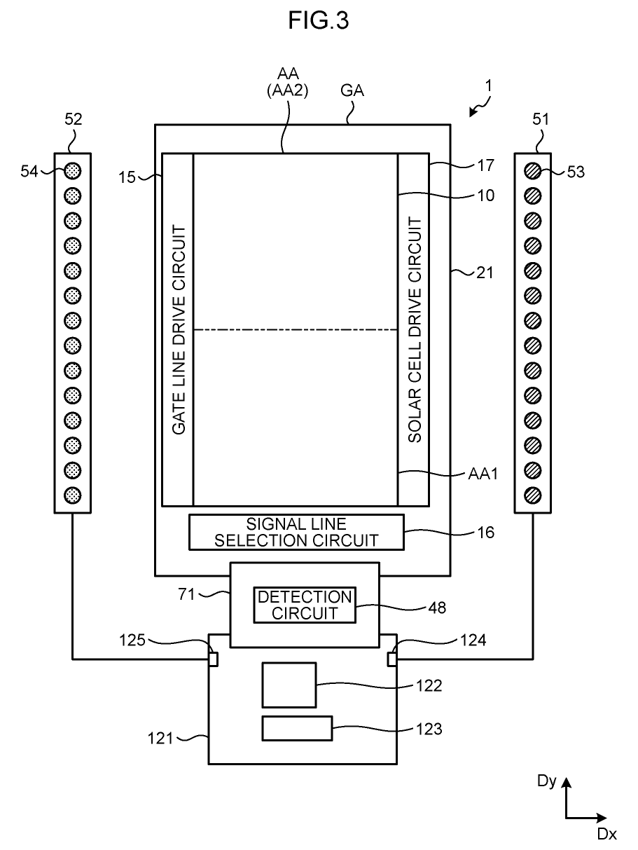

As illustrated in FIG. 2, the detection device 1 includes a housing 200, an optical sensor PD (organic optical sensor), a solar cell SC (organic photovoltaic device), a battery 73, and light sources 53 and 54. The housing 200 accommodates therein the optical sensor PD, the solar cell SC, the battery 73, and the light sources 53 and 54. FIG. 2 does not illustrate components (for example, various substrates such as a sensor substrate 21 (refer to FIG. 3)) other than the housing 200, the optical sensor PD, the solar cell SC, the battery 73, and the light sources 53 and 54.

The housing 200 is formed in a ring shape (annular shape) that can be worn on the object to be detected Fg, and is a wearable member to be worn on the living body. The housing 200 is formed of a housing material such as a synthetic resin. The housing 200 has a first part 201 having a light-transmitting inner peripheral surface 201a and a non-light-transmitting outer peripheral surface 201b, and a second part 202 having a light-transmitting outer peripheral surface 202b and a non-light-transmitting inner peripheral surface 202a. When the housing 200 is worn on the object to be detected Fg, the first part 201 faces a pad of the object to be detected Fg, and the second part 202 is located on the opposite side to the pad of the object to be detected Fg. The pad of the object to be detected Fg refers to a part of the object to be detected Fg that faces inward when a hand is closed. FIG. 2 illustrates portions of the housing 200 formed of a non- light-transmitting material with shading, and portions of the housing 200 formed of a light-transmitting material without shading.

Each of the first part 201 and the second part 202 is provided with at least one of the optical sensors PD and at least one of the solar cells SC. In the present embodiment, a plurality of the optical sensors PD and a plurality of the solar cells SC are formed so as to conform to the shape of the annular housing 200. In more detail, the optical sensors PD and the solar cells SC are provided in each of the first part 201 and the second part 202 of the annular housing 200.

The optical sensors PD provided in the first part 201 detects the biometric information on the living body from the object to be detected Fg (refer to FIG. 1). Specifically, light that has been emitted from the light sources 53 and 54 and transmitted through or reflected by the object to be detected Fg (refer to FIG. 1) passes through the light-transmitting inner peripheral surface 201a of the first part 201, and irradiates the optical sensors PD. Natural light incident on the optical sensors PD from outside is blocked by the non-light-transmitting outer peripheral surface 201b of the first part 201.

The solar cells SC provided in the second part 202 generate power using the natural light from outside. Specifically, the natural light from outside irradiates the solar cells SC through the light-transmitting outer peripheral surface 202b of the second part 202. The non- light-transmitting inner peripheral surface 202a of the second part 202 can inhibits the natural light from outside from traveling as stray light toward the object to be detected Fg and the first part 201. Alternatively, the non-light-transmitting inner peripheral surface 202a of the second part 202 blocks the light emitted from the light sources 53 and 54 and incident on the solar cells SC in the second part 202.

In the first part 201, the total area of at least one optical sensor PD is larger than the total area of at least one solar cell SC. In the second part 202, the total area of at least one optical sensor PD is smaller than the total area of at least one solar cell SC. In the present embodiment, the total area of the optical sensors PD is larger than the total area of the solar cells SC in the first part 201, and the total area of the optical sensors PD is smaller than the total area of the solar cells SC in the second part 202. This configuration allows the optical sensors PD provided in the first part 201 to have higher detection sensitivity than those in the second part 202 and detect the biometric information on the object to be detected Fg (refer to FIG. 1) satisfactorily. This configuration also allows the solar cells SC provided in the second part 202 to have a higher power generation efficiency than those in the first part 201 and generate the power based on the natural light from outside satisfactorily. The optical sensors PD may be arranged in the first part 201, and the solar cells SC may be arranged in the second part 202.

The light sources 53 and 54 are provided at locations overlapping neither the optical sensors PD nor the solar cells SC inside the housing 200. The locations of the light sources 53 and 54 illustrated in FIG. 2 are merely exemplary and can be changed as appropriate. That is, the light sources 53 and 54 may be arranged at any locations as long as the light emitted from the light sources 53 and 54 and transmitted through or reflected by the object to be detected Fg appropriately irradiates the optical sensors PD.

The battery 73 is a secondary battery that can be repeatedly charged and discharged. The battery 73 is a film lithium-ion battery, for example. The battery 73 is charged with the power generated by the solar cells SC. The battery 73 supplies the charged power to various parts that require power in the detection of the optical sensors PD. The battery 73 supplies the power to the light sources 53 and 54, for example. The battery 73 is located between the outer peripheral surface 201b of the first part 201 and some of the optical sensors PD and some of the solar cells SC. The battery 73 may be located at any location as long as the detection of the optical sensors PD and the power generation of the solar cells SC are not hindered, specifically, the light from the light sources 53 and 54 and the natural light from outside are not blocked.

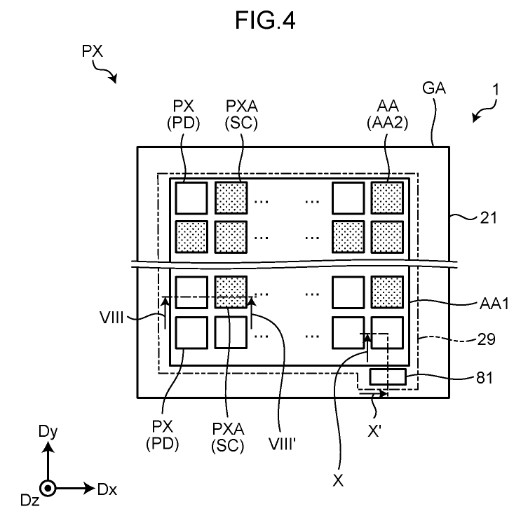

FIG. 3 is a plan view schematically illustrating the detection device according to the embodiment. FIG. 4 is a plan view schematically illustrating an arrangement example of the optical sensors and the solar cells in the detection area. FIGS. 3 and 4 are plan views each schematically illustrating a state in which the sensor substrate 21 before being accommodated in the housing 200 is developed in a planar shape.

As illustrated in FIG. 3, the detection device 1 includes the sensor substrate 21 (substrate), a sensor 10, a gate line drive circuit 15, a signal line selection circuit 16, a solar cell drive circuit 17, a detection circuit 48, a control circuit 122, a power supply circuit 123, a first light source base member 51, a second light source base member 52, and the light sources 53 and 54. The first light source base member 51 is provided with a plurality of the light sources 53. The second light source base member 52 is provided with a plurality of the light sources 54.

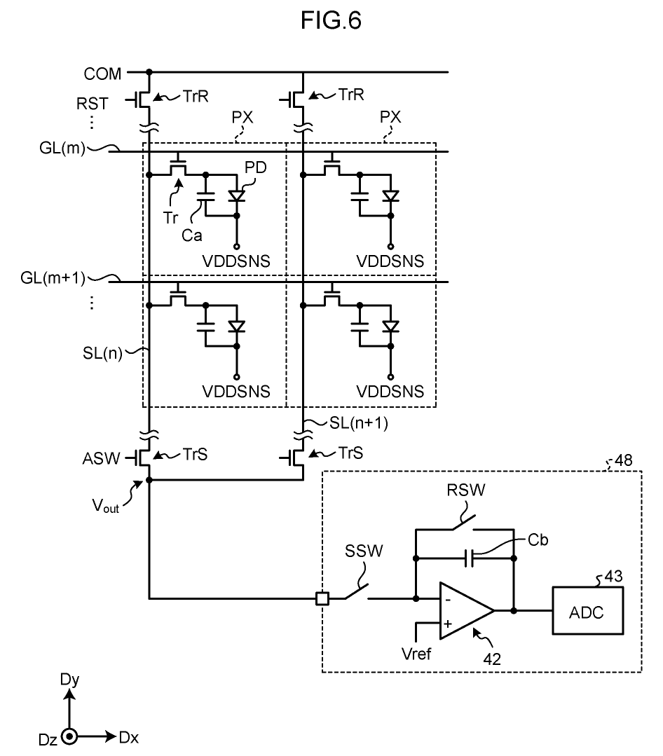

The sensor substrate 21 is electrically coupled to a control substrate 121 via a wiring substrate 71. The wiring substrate 71 and the control substrate 121 are flexible printed circuit boards, for example. The wiring substrate 71 is provided with the detection circuit 48. The control substrate 121 is provided with the control circuit 122 and the power supply circuit 123. The control circuit 122 is a field-programmable gate array (FPGA), for example. The control circuit 122 supplies control signals to the sensor 10, the gate line drive circuit 15, the signal line selection circuit 16, and the solar cell drive circuit 17 to control detection operations of the sensor 10. The control circuit 122 supplies control signals to the light sources 53 and 54 to control lighting and non- lighting of the light sources 53 and 54. The power supply circuit 123 supplies voltage signals including, for example, a sensor power supply signal (sensor power supply voltage) VDDSNS (refer to FIG. 6) to the sensor 10, the gate line drive circuit 15, the signal line selection circuit 16, and the solar cell drive circuit 17. The power supply circuit 123 supplies a power supply voltage to the light sources 53 and 54.

The sensor substrate 21 has a detection area AA and a peripheral area GA. The detection area AA is an area provided with the optical sensors PD and the solar cells SC (refer to FIG. 4) included in the sensor 10. The peripheral area GA is an area between the outer perimeter of the detection area AA and the outer edges of the sensor substrate 21 and is an area provided with neither the optical sensors PD nor the solar cells SC.

The detection area AA includes a first detection area AA1 and a second detection area AA2. The first detection area AA1 and the second detection area AA2 are provided adjacent to each other in a second direction Dy. The first detection area AA1 is located in the first part 201 of the housing 200, and the second detection area AA2 is located in the second part 202 of the housing 200. That is, the second direction Dy of the sensor substrate 21 extends along the circumferential direction of the housing 200.

In the following description, a first direction Dx is one direction in a plane parallel to the sensor substrate 21. The second direction Dy is one direction in the plane parallel to the sensor substrate 21 and is a direction orthogonal to the first direction Dx. The second direction Dy may, however, non-orthogonally intersect the first direction Dx. A third direction Dz is a direction orthogonal to the first direction Dx and the second direction Dy and is a direction normal to a principal surface of the sensor substrate 21. The term "plan view" refers to a positional relation when viewed from a direction orthogonal to the sensor substrate 21 in a state in which the sensor substrate 21 is developed in a planar shape.

As illustrated in FIG. 4, a plurality of sensor pixels PX and a plurality of solar cell pixels PXA are arranged in a matrix having a row-column configuration in the detection area AA. Each of the sensor pixels PX includes the optical sensor PD. Each of the solar cell pixels PXA includes the solar cell SC. The arrangement density (area) of the sensor pixels PX and the arrangement density (area) of the solar cell pixels PXA in the first detection area AA1 differ from the arrangement density (area) of the sensor pixels PX and the arrangement density (area) of the solar cell pixels PXA in the second detection area AA2.

More specifically, when four pixels in two rows and two columns are assumed as a reference unit, one reference unit includes three sensor pixels PX (optical sensors PD) and one solar cell pixel PXA (solar cell SC) in the first detection area AA1. One reference unit includes one sensor pixel PX (optical sensor PD) and three solar cell pixels PXA (solar cells SC) in the second detection area AA2. With this configuration, when the sensor substrate 21 is accommodated in the housing 200, the total area of the optical sensors PD is larger than the total area of the solar cells SC in the first part 201, and the total area of the optical sensors PD is smaller than the total area of the solar cells SC in the second part 202, as described above.

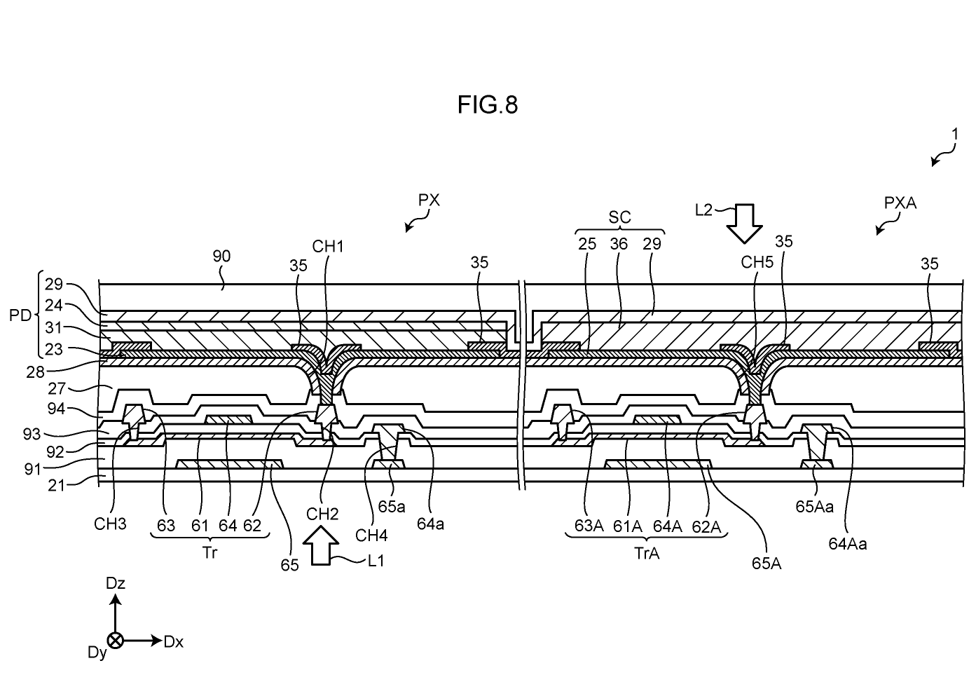

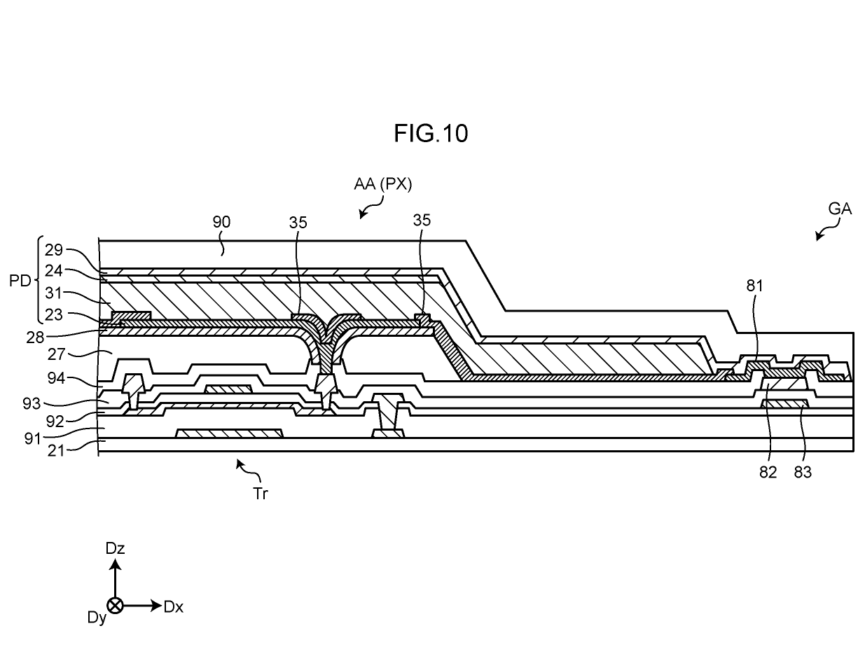

A common electrode 29 and a common electrode coupling terminal 81 illustrated in FIG. 4 will be described later with reference to FIGS. 8 and 10.

Referring back to FIG. 3, the gate line drive circuit 15, the signal line selection circuit 16, and the solar cell drive circuit 17 are provided in the peripheral area GA. Specifically, the gate line drive circuit 15 and the solar cell drive circuit 17 are provided in an area extending along the second direction Dy in the peripheral area GA. The signal line selection circuit 16 is provided in an area extending along the first direction Dx in the peripheral area GA, and is provided between the sensor 10 and the detection circuit 48.

The light sources 53 are provided on the first light source base member 51 and are arranged along the second direction Dy. The light sources 54 are provided on the second light source base member 52 and are arranged along the second direction Dy. The first light source base member 51 and the second light source base member 52 are electrically coupled to the control circuit 122 and the power supply circuit 123 through respective terminals 124 and 125 provided on the control substrate 121.

For example, inorganic light-emitting diodes (LEDs) or organic electroluminescent (EL) diodes (organic light- emitting diodes (OLEDs)) are used as the light sources 53 and 54. The light sources 53 and 54 emit light having different wavelengths from each other.

First light emitted from the light sources 53 is mainly reflected on a surface of the object to be detected, such as the finger, and enters the sensor 10. As a result, the sensor 10 can detect a fingerprint by detecting a shape of asperities on the surface of the finger or the like. Second light emitted from the light sources 54 is mainly reflected in the finger or the like, or transmitted through the finger or the like, and enters the sensor 10. As a result, the sensor 10 can detect the information on the living body in the finger or the like. Examples of the information on the living body include, but are not limited to, pulse waves, pulsation, and a vascular image of the finger or a palm. That is, the detection device 1 may be configured as a fingerprint detection device to detect the fingerprint or a vein detection device to detect a vascular pattern of, for example, veins.

The arrangement of the light sources 53 and 54 illustrated in FIG. 3 is merely an example, and can be changed as appropriate. The detection device 1 is provided with a plurality of types of the light sources 53 and 54 as light sources. However, the light sources are not limited thereto, and may be of one type. For example, the light sources 53 and 54 may be arranged on each of the first and the second light source base members 51 and 52. The light sources 53 and 54 may be provided on one light source base member, or three or more light source base members. Alternatively, only at least one light source needs to be disposed.

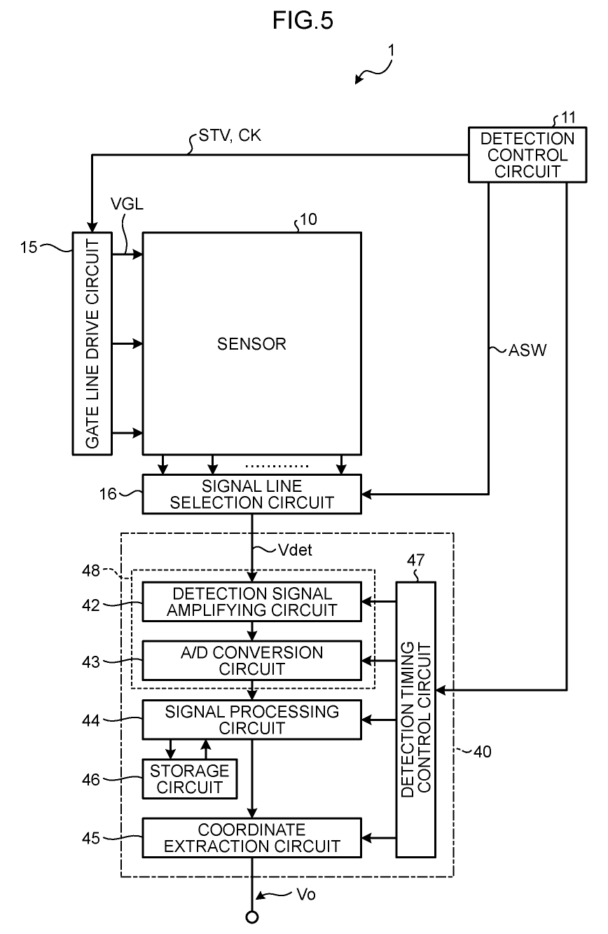

FIG. 5 is a block diagram illustrating a configuration example of the detection device according to the embodiment. As illustrated in FIG. 5, the detection device 1 further includes a detection control circuit 11 and a detector (detection signal processing circuit) 40. The control circuit 122 includes one, some, or all functions of the detection control circuit 11. The control circuit 122 also includes one, some, or all functions of the detector 40 other than those of the detection circuit 48.

The sensor 10 includes the optical sensors PD. Each of the optical sensors PD included in the sensor 10 outputs an electrical signal corresponding to light irradiating the optical sensor PD as a detection signal Vdet to the signal line selection circuit 16. The sensor 10 performs the detection in response to a gate drive signal VGL supplied from the gate line drive circuit 15.

The detection control circuit 11 is a circuit that supplies respective control signals to the gate line drive circuit 15, the signal line selection circuit 16, and the detector 40 to control operations of these components. The detection control circuit 11 supplies various control signals including, for example, a start signal STV and a clock signal CK to the gate line drive circuit 15. The detection control circuit 11 also supplies various control signals including, for example, a selection signal ASW to the signal line selection circuit 16. The detection control circuit 11 also supplies various control signals to the light sources 53 and 54 to control the lighting and non-lighting of the respective light sources 53 and 54.

The gate line drive circuit 15 is a circuit that drives a plurality of gate lines GL (refer to FIG. 6) based on the various control signals. The gate line drive circuit 15 sequentially or simultaneously selects the gate lines GL, and supplies the gate drive signals VGL to the selected gate lines GL. Through this operation, the gate line drive circuit 15 selects the optical sensors PD coupled to the gate lines GL.

The signal line selection circuit 16 is a switch circuit that sequentially or simultaneously selects a plurality of signal lines SL (refer to FIG. 6). The signal line selection circuit 16 is a multiplexer, for example. The signal line selection circuit 16 couples the selected signal lines SL to the detection circuit 48 based on the selection signal ASW supplied from the detection control circuit 11. Through this operation, the signal line selection circuit 16 outputs the detection signals Vdet of the optical sensors PD to the detector 40.

The detector 40 includes the detection circuit 48, a signal processing circuit 44, a coordinate extraction circuit 45, a storage circuit 46, and a detection timing control circuit 47. The detection timing control circuit 47 controls the detection circuit 48, the signal processing circuit 44, and the coordinate extraction circuit 45 to operate synchronously based on a control signal supplied from the detection control circuit 11.

The detection circuit 48 is an analog front-end (AFE) circuit, for example. The detection circuit 48 is a signal processing circuit having functions of at least a detection signal amplifying circuit 42 and an analog-to-digital (A/D) conversion circuit 43. The detection signal amplifying circuit 42 amplifies the detection signals Vdet. The A/D conversion circuit 43 converts analog signals output from the detection signal amplifying circuit 42 into digital signals.

The signal processing circuit 44 is a logic circuit that detects predetermined physical quantities received by the sensor 10 based on output signals of the detection circuit 48. The signal processing circuit 44 can detect the asperities on the surface of the finger or the palm based on the signals from the detection circuit 48 when the finger is in contact with or in proximity to a detection surface. The signal processing circuit 44 can also detect the information on the living body based on the signals from the detection circuit 48. Examples of the information on the living body include, but are not limited to, the vascular image, the pulse waves, the pulsation, and a blood oxygen level of the finger or the palm.

The storage circuit 46 temporarily stores therein signals calculated by the signal processing circuit 44. The storage circuit 46 may be, for example, a random-access memory (RAM) or a register circuit.

The coordinate extraction circuit 45 is a logic circuit that obtains detected coordinates of the asperities on the surface of the finger or the like when the contact or proximity of the finger is detected by the signal processing circuit 44. The coordinate extraction circuit 45 is the logic circuit that also obtains detected coordinates of blood vessels in the finger or the palm. The coordinate extraction circuit 45 combines the detection signals Vdet output from the respective optical sensors PD of the sensor 10 to generate two-dimensional information indicating the shape of the asperities on the surface of the finger or the like and two-dimensional information indicating the shape of the blood vessels in the finger or the palm. The coordinate extraction circuit 45 may output the detection signals Vdet as sensor output voltages Vo instead of calculating the detected coordinates.

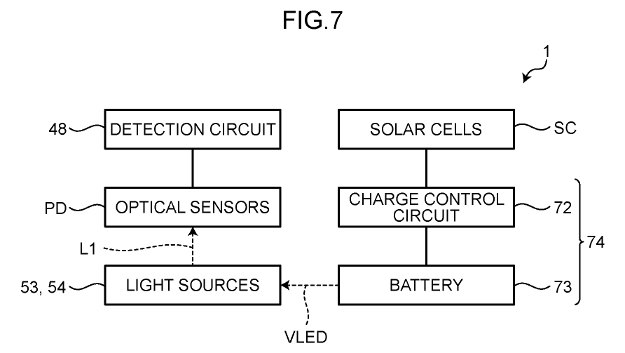

FIG. 6 is a circuit diagram illustrating the detection device according to the embodiment. FIG. 6 also illustrates a circuit configuration of the detection circuit 48. FIG. 6 also illustrates only the sensor pixels PX and the detection circuit 48 while omitting the solar cells SC. As illustrated in FIG. 7, the solar cells SC charge the battery 73 through another route via a charge control circuit 72. As illustrated in FIG. 6, the sensor pixel PX includes the optical sensor PD, a capacitive element Ca, and a drive transistor Tr. The capacitive element Ca is capacitance (sensor capacitance) generated in the optical sensor PD, and is equivalently coupled in parallel to the optical sensor PD.

FIG. 6 illustrates two gate lines GL(m) and GL(m+1) arranged in the second direction Dy among the gate lines GL. FIG. 6 also illustrates two signal lines SL(n) and SL(n+1) arranged in the first direction Dx among the signal lines SL. The sensor pixel PX is an area surrounded by the gate lines GL and the signal lines SL.

The gate lines GL each extend in the first direction Dx and are arranged with gaps interposed therebetween in the second direction Dy. The signal lines SL each extend in the second direction Dy and are arranged with gaps interposed therebetween in the first direction Dx. The sensor pixels PX (optical sensors PD) are each provided in an area surrounded by two of the gate lines GL and two of the signal lines SL.

The drive transistors Tr are provided correspondingly to the optical sensors PD. Each of the drive transistors Tr is configured as a thin-film transistor, and in this example, configured as an n-channel metal oxide semiconductor (MOS) thin-film transistor (TFT).

Each of the gate lines GL is coupled to the gates of the drive transistors Tr arranged in the first direction Dx. Each of the signal lines SL is coupled to either the sources or the drains of the drive transistors Tr arranged in the second direction Dy. The other of the sources and the drains of the drive transistors Tr are each coupled to the anode of the optical sensor PD and the capacitive element Ca.

The cathode of the optical sensor PD is supplied with the sensor power supply signal VDDSNS from the power supply circuit 123 (refer to FIG. 1). The signal line SL and the capacitive element Ca are supplied with a sensor reference voltage COM serving as an initial potential of the signal line SL and the capacitive element Ca from the power supply circuit 123 via a reset transistor TrR.

When the sensor pixel PX is irradiated with light in an exposure period, a current corresponding to the amount of the light flows through the optical sensor PD. As a result, an electric charge is stored in the capacitive element Ca. When the drive transistor Tr is turned on in a readout period, a current corresponding to the electric charge stored in the capacitive element Ca flows through the signal line SL. The signal line SL is coupled to the detection circuit 48 via an output transistor TrS of the signal line selection circuit 16. Thus, the detection device 1 can detect a signal corresponding to the amount of the light irradiating the optical sensor PD for each of the sensor pixels PX.

During the readout period, a switch SSW is turned on to couple the detection circuit 48 to the signal line SL. The detection signal amplifying circuit 42 of the detection circuit 48 converts a current supplied from the signal line SL into a variation of a voltage corresponding to the value of the current, and amplifies the result. A reference potential (Vref) having a fixed potential is supplied to a non-inverting input portion (+) of the detection signal amplifying circuit 42, and the signal line SL is coupled to an inverting input portion (-) of the detection signal amplifying circuit 42. In the embodiment, the same signal as the sensor reference voltage COM is supplied as the reference potential (Vref) voltage. The control circuit 122 (refer to FIG. 1) calculates the difference between the detection signal Vdet when light irradiates the optical sensor PD and the detection signal Vdet when light does not irradiate the optical sensor PD, as each of the sensor output voltages Vo. The detection signal amplifying circuit 42 includes a capacitive element Cb and a reset switch RSW. During a reset period, the reset switch RSW is turned on to reset the electric charge of the capacitive element Cb.

The drive transistor Tr is not limited to the n-type TFT and may be configured as a p-type TFT. The pixel circuit of the sensor pixel PX illustrated in FIG. 6 is merely exemplary. The sensor pixel PX may include one photodiode PD provided with a plurality of transistors.

FIG. 7 is a block diagram schematically illustrating a configuration example of the optical sensors, the solar cells, a battery circuit, and the light sources. As illustrated in FIG. 7, the detection device 1 includes the detection circuit 48 coupled to the optical sensors PD, and a battery circuit 74 coupled to the solar cells SC. The detection circuit 48 has been described with reference to FIGS. 5 and 6, and therefore, will not be described again.

The battery circuit 74 includes the charge control circuit 72 and the battery 73. The charge control circuit 72 is a circuit that adjusts the power supplied from the solar cells SC to control the charging of the battery 73. For example, the charge control circuit 72 is coupled between a coupling transistor TrA (refer to FIG. 8) and the solar cells SC and adjusts the power to charge the battery 73. Alternatively, the charge control circuit 72 controls the operation of the solar cell drive circuit 17 (refer to FIG. 3) to control the operation of the coupling transistor TrA, thereby adjusting the power to be supplied from the solar cells SC to the battery 73. In this case, the coupling transistor TrA may be directly coupled to the battery 73. The charge control circuit 72 appropriately adjusts the value of the voltage and the value of the current that are output to the battery 73 according to the state (capacity, temperature, and other factors) of the battery 73. When the detection operation is performed by the optical sensors PD and the detection circuit 48, the coupling transistor TrA may be turned off to stop charging the battery 73, and when the detection operation is not performed, the coupling transistor TrA may be turned on to charge the battery 73.

The battery 73 supplies a drive voltage VLED to the light sources 53 and 54. The drive voltage VLED causes the light sources 53 and 54 to emit light Li. The optical sensor PD outputs the electrical signal corresponding to the emitted light Li as the detection signal Vdet. The detection circuit 48 processes the detection signal Vdet from the optical sensor PD as described above.

While the example has been described in which the battery 73 supplies the drive voltage VLED to the light sources 53 and 54, the battery 73 may be used for supplying power to other components and circuits included in the detection device 1.

The following describes a configuration of the optical sensor PD and the solar cell SC. FIG. 8 is a sectional view taken along section VIII-VIII' of FIG. 4. In the following description, a direction from the sensor substrate 21 toward a sealing film 90 in a direction orthogonal to a surface of the sensor substrate 21 is referred to as an "upper side" or simply "above". A direction from the sealing film 90 toward the sensor substrate 21 is referred to as a "lower side" or simply "below".

As illustrated in FIG. 8, in the detection device 1, a TFT layer, an organic insulating film 27, an inorganic insulating film 28, the optical sensor PD, the solar cell SC, and the sealing film 90 are stacked in this order on the sensor substrate 21. The TFT layer is a circuit forming layer provided with the drive transistor Tr, the coupling transistor TrA, and various types of wiring such as the gate line GL and the signal line SL.

The sensor substrate 21 is an insulating substrate formed of a film-like resin. The drive transistor Tr provided in the TFT layer is provided in an area overlapping a first lower electrode 23 of the optical sensor PD. Specifically, the drive transistor Tr includes a semiconductor layer 61, a source electrode 62, a drain electrode 63, and a gate electrode 64.

The coupling transistor TrA is provided in an area overlapping a second lower electrode 25 of the solar cell SC. Specifically, the coupling transistor TrA includes a semiconductor layer 61A, a source electrode 62A, a drain electrode 63A, and a gate electrode 64A.

While the multilayered configuration of the drive transistor Tr is described with reference to FIG. 8, the multilayered configuration of the coupling transistor TrA is the same as that of the drive transistor Tr, and the description of the drive transistor Tr can also be applied to the coupling transistor TrA.

The TFT layer includes an undercoat film 91, a gate insulating film 92, an interlayer insulating film 93, and an overlay insulating film 94, as insulating films.

A light-blocking film 65 is provided on the sensor substrate 21. The light-blocking film 65 is provided between the semiconductor layer 61 and the sensor substrate 21. The light-blocking film 65 can reduce light entering a channel region of the semiconductor layer 61 from the sensor substrate 21 side.

The undercoat film 91 is provided on the sensor substrate 21 so as to cover the light-blocking film 65. The undercoat film 91 is formed, for example, of an inorganic insulating film such as a silicon nitride film or a silicon oxide film. The configuration of the undercoat film 91 is not limited to that illustrated in FIG. 8. For example, the undercoat film 91 may be a multilayered film having two, three, or more layers.

The drive transistor Tr is provided on the sensor substrate 21. The semiconductor layer 61 is provided on the undercoat film 91. The gate insulating film 92 is provided on the undercoat film 91 so as to cover the semiconductor layer 61. The gate insulating film 92 is, for example, an inorganic insulating film such as a silicon oxide film. The gate electrode 64 is provided on the gate insulating film 92.

In the example illustrated in FIG 8 , the drive transistor Tr has a top-gate structure. However, the drive transistor Tr is not limited thereto and may have a bottom- gate structure or a dual-gate structure in which the gate electrodes 64 are provided on the upper and lower sides of the semiconductor layer 61.

Coupling wiring 64a is provided in the same layer as the gate electrode 64. The coupling wiring 64a is electrically coupled to the gate electrode 64. Coupling wiring 65a is provided in the same layer as the light- blocking film 65. The coupling wiring 65a is electrically coupled to the light-blocking film 65. The coupling wiring 64a is coupled to the coupling wiring 65a through a contact hole CH4 penetrating the undercoat film 91 and the gate insulating film 92. As a result, the light-blocking film 65 is electrically coupled to the gate electrode 64 via the coupling wiring 64a and 65a and is supplied with the same potential as that of the gate electrode 64. A light- blocking film 65A and coupling wiring 64Aa and 65Aa provided in the coupling transistor TrA have the same configurations as those of the light-blocking film 65 and the coupling wiring 64a and 65a provided in the coupling transistor Tr.

The interlayer insulating film 93 is provided on the gate insulating film 92 so as to cover the gate electrode 64. The interlayer insulating film 93 has, for example, a multilayered structure of a silicon nitride film and a silicon oxide film. The source electrode 62 and the drain electrode 63 are provided on the interlayer insulating film 93. The source electrode 62 is coupled to a source region of the semiconductor layer 61 through a contact hole CH2 provided in the gate insulating film 92 and the interlayer insulating film 93. The drain electrode 63 is coupled to a drain region of the semiconductor layer 61 through a contact hole CH3 provided in the gate insulating film 92 and the interlayer insulating film 93. The overlay insulating film 94 is provided on the interlayer insulating film 93 so as to cover the source electrode 62 and the drain electrode 63.

The organic insulating film 27 is provided on the overlay insulating film 94 so as to cover the source electrode 62 and the drain electrode 63 of the drive transistor Tr. The organic insulating film 27 is a planarizing film formed of an organic insulating material. In the present embodiment, a contact hole CH1 in the organic insulating film 27 is provided in an area thereof overlapping the source electrode 62. The first lower electrode 23 of the optical sensor PD is electrically coupled to the source electrode 62 at the bottom of the contact hole CH1.

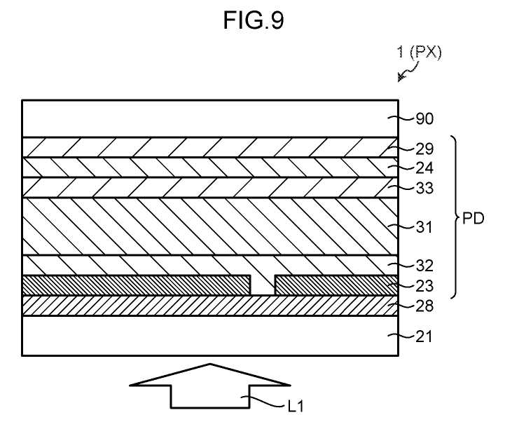

The inorganic insulating film 28 is provided on the organic insulating film 27. The inorganic insulating film 28 is, for example, a barrier film formed of an inorganic insulating material, such as a silicon nitride (SiN) film. The optical sensor PD and the solar cell SC are provided on the inorganic insulating film 28. The following describes a detailed configuration of the optical sensor PD with reference to FIGS. 8 and 9. FIG. 9 is an enlarged sectional view illustrating the optical sensor in FIG. 8. FIG. 8 does not illustrate a first lower buffer layer 32 and a first upper buffer layer 33 of the optical sensor PD.

As illustrated in FIGS. 8 and 9, the optical sensor PD includes the first lower electrode 23, the first lower buffer layer 32, a first active layer 31, the first upper buffer layer 33, a first upper electrode 24, and the common electrode 29. In the optical sensor PD, the first lower electrode 23, the first lower buffer layer 32 (hole transport layer), the first active layer 31, the first upper buffer layer 33 (electron transport layer), the first upper electrode 24, and the common electrode 29 are stacked in this order in a direction orthogonal to the sensor substrate 21. The optical sensor PD of the present embodiment is an organic photodiode (OPD) using an organic semiconductor as the first active layer 31.

The first lower electrode 23 is an anode electrode of the optical sensor PD and is formed of a light-transmitting conductive material such as indium tin oxide (ITO). The first lower electrodes 23 are separated from each other so as to correspond to the optical sensors PD. The first lower buffer layer 32, the first active layer 31, the first upper buffer layer 33, and the first upper electrode 24 are continuously provided across the optical sensors PD. Specifically, the first lower buffer layer 32, the first active layer 31, the first upper buffer layer 33, and the first upper electrode 24 are provided so as to overlap the first lower electrodes 23 of the adjacent optical sensors PD, and provided so as to overlap also an insulating film 35 between the adjacent optical sensors PD.

The insulating film 35 is provided so as to cover the periphery of the first lower electrode 23. Although not illustrated, the insulating film 35 is provided on the inorganic insulating film 28, between the first lower electrodes 23 of the adjacent optical sensors PD. The insulating film 35 insulates the first lower electrodes 23 of the adjacent optical sensors PD from each other. The insulating film 35 is provided so as to cover the contact hole CH1 and covers the first lower electrode 23 in an area thereof overlapping the contact hole CH1. With this configuration, even if a step break occurs in the first lower buffer layer 32 (hole transport layer) in the contact hole CH1, the insulating film 35 can reduce an occurrence of a short circuit between the first active layer 31 and the first lower electrode 23. In the present embodiment, the insulating film 35 is formed of an inorganic insulating material, such as a silicon nitride (SiN) film or a silicon oxide (SiO2) film.

The contact hole CH1 is provided so as to penetrate the organic insulating film 27 in the thickness direction thereof (third direction Dz) at the center of the first lower electrode 23. The first lower electrode 23 is coupled to the source electrode 62 at the bottom of the contact hole CH1. The position of the contact hole CH1, that is, the coupling point between the optical sensor PD and the drive transistor Tr, is not limited to the center of the first lower electrode 23, and can be changed as appropriate.

The first active layer 31 changes in characteristics (for example, voltage-current characteristics and resistance value) according to light emitted thereto. An organic material is used as a material of the first active layer 31. Specifically, the first active layer 31 has a bulk heterostructure containing a mixture of a p-channel organic semiconductor and an n-channel fullerene derivative ((6,6)-phenyl-C6i-butyric acid methyl ester (PCBM)) that is an n-channel organic semiconductor. As the first active layer 31, low-molecular-weight organic materials can be used including, for example, fullerene (C6o), phenyl-C6i- butyric acid methyl ester (PCBM), copper phthalocyanine (CuPc), fluorinated copper phthalocyanine (Fi6CuPc), 5,6,11,12-tetraphenyltetracene (rubrene), and perylene diimide (PDI) (derivative of perylene).

The first active layer 31 can be formed by a vapor deposition process (dry process) using any of the low- molecular-weight organic materials listed above. In this case, the first active layer 31 may be, for example, a multilayered film of CuPc and Fi6CuPc, or a multilayered film of rubrene and Cho. The first active layer 31 can also be formed by a coating process (wet process). In this case, the first active layer 31 is made using a material obtained by combining any of the above-listed low- molecular-weight organic materials with a high-molecular- weight organic material. As the high-molecular-weight organic material, for example, poly(3-hexylthiophene) (P3HT) and F8-alt-benzothiadiazole (F8BT) can be used. The first active layer 31 can be a film made of a mixture of P3HT and PCBM, or a film made of a mixture of F8BT and PDI.

The first lower buffer layer 32 is a hole transport layer, and the first upper buffer layer 33 is an electron transport layer. The first lower buffer layer 32 and the first upper buffer layer 33 are provided to facilitate holes and electrons generated in the first active layer 31 to reach the first lower electrode 23 or the first upper electrode 24. The first lower buffer layer 32 (hole transport layer) is in direct contact with the top of the first lower electrode 23 and is also provided on the insulating film 35 between the adjacent first lower electrodes 23. The first active layer 31 is in direct contact with the top of the first lower buffer layer 32. The material of the hole transport layer is a metal oxide layer. Tungsten oxide (WO3), molybdenum oxide, or the like is used as the oxide metal layer.

The first upper buffer layer 33 (electron transport layer) is in direct contact with the top of the first active layer 31, and the first upper electrode 24 is in direct contact with the top of the first upper buffer layer 33. Polyethylenimine ethoxylated (PEIE) is used as a material of the electron transport layer.

The materials and the manufacturing methods of the first lower buffer layer 32, the first active layer 31, and the first upper buffer layer 33 are merely exemplary, and other materials and manufacturing methods may be used. For example, each of the first lower buffer layer 32 and the first upper buffer layer 33 is not limited to a single- layer film, and may be formed as a multilayered film that includes an electron block layer and a hole block layer.

The first upper electrode 24 is provided on the first upper buffer layer 33. The first upper electrode 24 is a cathode electrode of the optical sensors PD and is continuously formed over the entire detection area AA. In other words, the first upper electrode 24 is continuously provided on the optical sensors PD. The first upper electrode 24 faces the first lower electrodes 23 with the first lower buffer layer 32, the first active layer 31, and the first upper buffer layer 33 interposed therebetween. The first upper electrode 24 is formed, for example, of a non-light-transmitting conductive material such as silver (Ag).

The common electrode 29 is provided on the first upper electrode 24. The common electrode 29 is continuously provided across the optical sensors PD and the solar cells SC. The common electrode 29 is formed, for example, of a light-transmitting conductive material such as ITO. The optical sensors PD and the solar cells SC are supplied with a common reference potential via the common electrode 29.

FIG. 10 is a sectional view taken along section X-X' of FIG. 4. As illustrated in FIGS. 4 and 10, the common electrode coupling terminal 81 is provided in the peripheral area GA of the sensor substrate 21. The common electrode 29 is continuously provided from the detection area AA to the peripheral area GA, and is coupled to the common electrode coupling terminal 81 in the peripheral area GA.

In more detail, as illustrated in FIG. 10, the common electrode coupling terminal 81 is provided on the overlay insulating film 94 in the peripheral area GA. Reference potential supply wiring 82 and 83 is provided between the layers of the common electrode coupling terminal 81 and the sensor substrate 21. The common electrode coupling terminal 81 is coupled to the reference potential supply wiring 82 and is supplied with the reference potential.

In an area between the common electrode coupling terminal 81 and the sensor pixel PX, the organic insulating film 27 and the inorganic insulating film 28 are removed, and the insulating film 35, the first active layer 31, the common electrode 29, and the sealing film 90 are stacked in this order on the overlay insulating film 94. In an area provided with the common electrode coupling terminal 81, neither the insulating film 35 nor the first active layer 31 is provided, and the common electrode 29 and the sealing film 90 are stacked in this order on the common electrode coupling terminal 81. With this configuration, the common electrode 29 is coupled to the common electrode coupling terminal 81 and is supplied with the reference potential from the reference potential supply wiring 82 and 83.

Referring back to FIGS. 8 and 9, the sealing film 90 is provided on the common electrode 29 and continuously provided so as to cover the optical sensors PD and the solar cells SC. An inorganic film such as a silicon nitride film or an aluminum oxide film, or a resin film such as an acrylic film is used as the sealing film 90. The sealing film 90 is not limited to a single layer, and may be a multilayered film having two or more layers obtained by combining the inorganic film with the resin film mentioned above. The sealing film 90 well seals the optical sensors PD and the solar cells SC, and thus can reduce moisture entering the optical sensors PD and the solar cells SC from the upper surface side thereof.

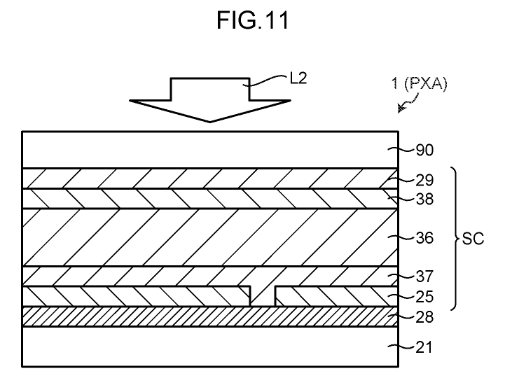

The following describes a detailed configuration of the solar cell SC with reference to FIGS. 8 and 11. FIG. 11 is an enlarged sectional view illustrating the solar cell in FIG. 8. FIG. 8 does not illustrate a second lower buffer layer 37 and a second upper buffer layer 38 of the solar cell SC.

As illustrated in FIGS. 8 and 11, the solar cell SC is a photodiode (OPD) that has a configuration similar to that of the optical sensor PD and uses an organic semiconductor as a second active layer 36. Specifically, the solar cell SC include the second lower electrode 25, the second lower buffer layer 37, the second active layer 36, the second upper buffer layer 38, and the common electrode 29. In the solar cell SC, the second lower electrode 25, the second lower buffer layer 37 (hole transport layer), the second active layer 36, the second upper buffer layer 38 (electron transport layer), and the common electrode 29 are stacked in this order in the direction orthogonal to the sensor substrate 21.

The second lower electrode 25, the second lower buffer layer 37 (hole transport layer), the second active layer 36, and the second upper buffer layer 38 of the solar cell SC are provided in the same layer as the first lower electrode 23, the first lower buffer layer 32, the first active layer 31, and the first upper buffer layer 33 of the optical sensor PD, respectively.

The first lower electrode 23, the first lower buffer layer 32, the first active layer 31, the first upper buffer layer 33, and the first upper electrode 24 of the optical sensor PD are provided so as to be separated from the second lower electrode 25, the second lower buffer layer 37, the second active layer 36, and the second upper buffer layer 38 of the solar cell SC. The common electrode 29 and the sealing film 90 are provided between the optical sensor PD and the solar cell SC adjacent to each other. The common electrode 29 is provided so as to cover side surfaces of the first active layer 31 and the first upper electrode 24 of the optical sensor PD, and a side surface of the second active layer 36 of the solar cell SC. The sealing film 90 is provided so as to cover the common electrode 29 formed in a recessed shape between the optical sensor PD and the solar cell SC adjacent to each other.

The second lower electrode 25 of the solar cell SC is formed, for example, of a non-light-transmitting conductive material such as silver (Ag). The second lower electrodes 25 are separated from each other so as to correspond to the solar cells SC.

The insulating film 35 is provided so as to cover the periphery of the second lower electrode 25. The insulating film 35 is provided on the inorganic insulating film 28 between the adjacent first and second lower electrodes 23 and 25. The first lower electrode 23 of the optical sensor PD and the second lower electrode 25 of the solar cell SC adjacent to each other are insulated from each other by the insulating film 35. The insulating film 35 is provided so as to cover a contact hole CH5, and covers the second lower electrode 25 in an area overlapping the contact hole CH5.

The second lower buffer layer 37, the second active layer 36, and the second upper buffer layer 38 of the solar cell SC are formed of the same materials as those of the first lower buffer layer 32, the first active layer 31, and the first upper buffer layer 33 of the optical sensor PD, respectively.

The common electrode 29 is provided on the second upper buffer layer 38. In other words, the common electrode 29 doubles as an upper electrode of the solar cell SC.

With the configuration described above, the first lower electrode 23 of the optical sensor PD has a light- transmitting property, and the first upper electrode 24 thereof does not have a light-transmitting property. That is, the optical sensor PD is configured as a bottom- illuminated optical sensor. The light Li emitted from the light sources 53 and 54 and transmitted through or reflected by the object to be detected Fg irradiates the first lower electrode 23 side of the optical sensor PD. The light Li passes through the sensor substrate 21 and the first lower electrode 23 of the optical sensor PD and irradiates the first active layer 31.

The second lower electrode 25 of the solar cell SC does not have a light-transmitting property, and the common electrode 29 thereof has a light-transmitting property. That is, the solar cell SC is configured as a top- illuminated solar cell. Natural light L2 from outside irradiates the common electrode 29 side of the solar cell SC. Specifically, the natural light L2 passes through the sealing film 90 and the common electrode 29 of the solar cell SC and irradiates the second active layer 36.

Thus, the light Li transmitted through or reflected by the object to be detected Fg irradiates the optical sensor PD, and the natural light L2 is blocked by the first upper electrode 24. The natural light L2 irradiates the solar cell SC, and the light Li transmitted through or reflected by the object to be detected Fg is blocked by the second lower electrode 25. Thus, the detection device 1 can detect the light and generate the electricity satisfactorily using the optical sensor PD and the solar cell SC that are provided on the same sensor substrate 21.

The optical sensor PD and the solar cell SC illustrated in FIG. 8 are accommodated in the housing 200 illustrated in FIG. 2to be configured as the detection device 1. In this case, the lower surface of the sensor substrate 21 is placed facing the inner peripheral surfaces 201a and 202a of the housing 200, and the common electrode 29 and the sealing film 90 are placed facing the outer peripheral surfaces 201b and 202b of the housing 200.

When the optical sensor PD and the solar cell SC provided on the same sensor substrate 21 are accommodated in the annular housing 200, the light Li transmitted through or reflected by the object to be detected Fg irradiates the optical sensor PD from the inner peripheral surfaces 201a and 202a side, and the natural light L2 irradiates the solar cell SC from the outer peripheral surfaces 201b and 202b side. Therefore, the detection device 1 can detect the light and generate the electricity satisfactorily even when being configured such that the annular housing 200 accommodates therein the optical sensor PD and the solar cell SC.

As described above, the outer peripheral surface 201b and the inner peripheral surface 202a of the housing 200 do not have a light-transmitting property. Therefore, at the optical sensor PD located in the first part 201, the natural light L2 is blocked by the first upper electrode 24 and the outer peripheral surface 201b of the housing 200. At the solar cell SC located in the second part 202, the light Li is blocked by the second lower electrode 25 and the inner peripheral surface 202a of the housing 200. Thus, the detection sensitivity of the optical sensor PD can be improved.

In the optical sensor PD, at least the first lower electrode 23 only needs to have a light-transmitting property. In the solar cell SC, at least the common electrode 29 only needs to have a light-transmitting property. That is, the first upper electrode 24 of the optical sensor PD and the second lower electrode 25 of the solar cell SC may also be formed of a light-transmitting conductive material, such as ITO. Even in this case, the detection sensitivity of the optical sensor PD and the power generation efficiency of the solar cell SC can be ensured by accommodating the optical sensor PD and the solar cell SC in the housing 200 as described above.

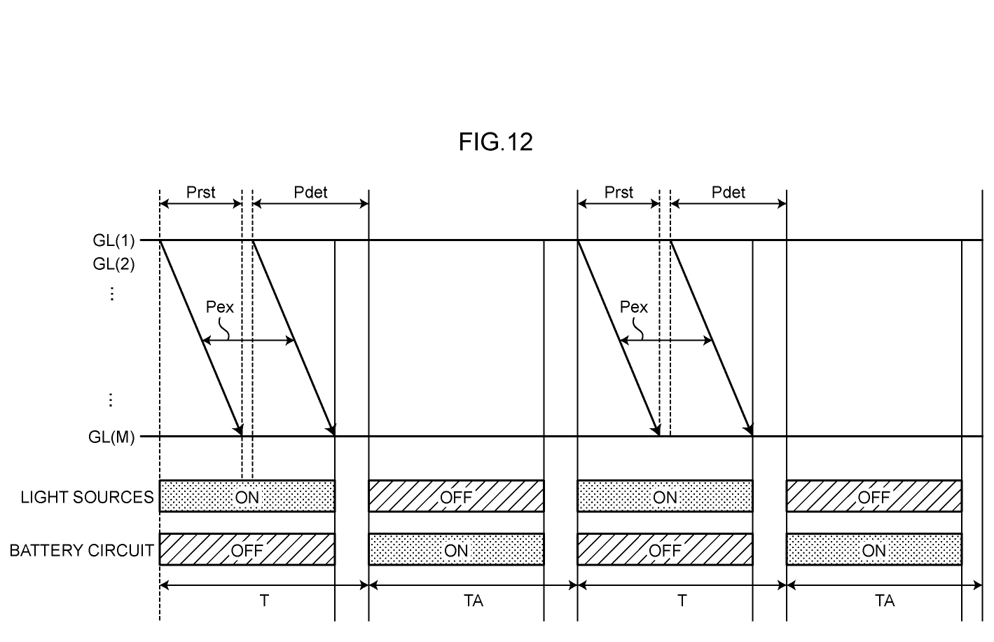

FIG. 12 is an explanatory diagram for explaining a relation between the detection by the optical sensors of the detection device and the operation of the solar cells. As illustrated in FIG. 12, in the detection device 1, a detection period T to perform the detection using the optical sensor PD and a charge period TA to charge the battery 73 using the solar cells SC are provided in a time- division manner. In FIG. 12, the detection period T and the charge period TA are alternately provided in such an order as the detection period T, the charge period TA, the detection period T, and the charge period TA.

In the detection period T, the detection device 1 executes a reset period Prst, an effective exposure period Pex, and a readout period Pdet. In the reset period Prst and the readout period Pdet, the gate line drive circuit 15 sequentially scans the gate lines from GL(1) to GL(M). That is, in the detection period T, the optical sensors PD in the entire detection area AA are scanned.

In the detection period T, the light sources 53 and 54 are turned on to emit the light Li. In the detection period T, the battery circuit 74 stops operating to stop charging the battery 73.

In the charge period TA, the solar cell drive circuit 17 turns on the coupling transistor TrA (into a conducting state), and a current generated in the solar cells SC and corresponding to the natural light L2 illuminating thereon is supplied to the battery circuit 74. In the battery circuit 74, the charge control circuit 72 charges the battery 73.

In the charge period TA, the gate line drive circuit 15 stops, and the light sources 53 and 54 are brought into a non-lighting state. That is, the optical sensors PD do not perform detection during the charge period TA.

Thus, the detection device 1 can improve the detection sensitivity of the optical sensors PD and can efficiently charge the battery 73 using the solar cells SC by executing the detection period T and the charge period TA in a time- division manner.

The operation example illustrated in FIG. 12 is merely exemplary. The detection by the optical sensors PD and the charging of the battery 73 by the solar cells SC can also be performed in the same period. While the detection period T and the charge period TA are alternately arranged, the periods (lengths) of the detection period T and the charge period TA may be changed as appropriate depending on the power generation state of the solar cells SC and the capacity of the battery 73.

### Modification

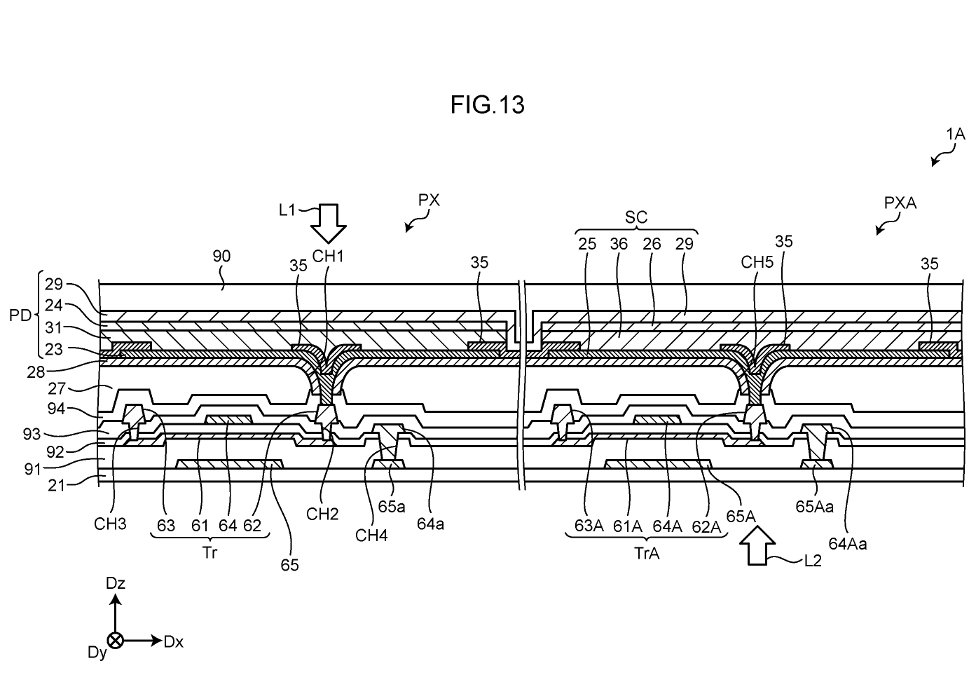

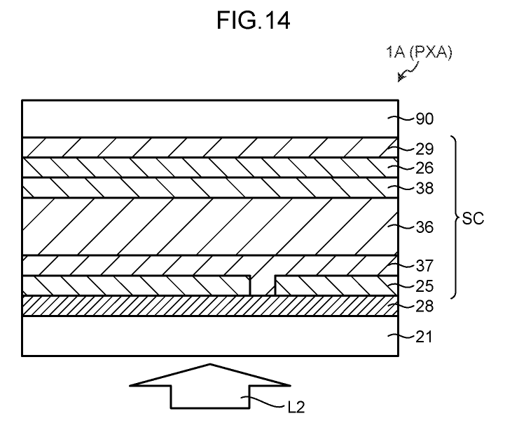

FIG. 13 is a sectional view of a detection device according to a modification of the embodiment. FIG. 14 is an enlarged sectional view illustrating the solar cell in FIG. 13. In the following description, the same components as those described in the embodiment described above are denoted by the same reference numerals, and the description thereof will not be repeated.

As illustrated in FIGS. 13 and 14, in a detection device lA according to the modification, the solar cell SC includes a second upper electrode 26. In more detail, in the solar cell SC, the second lower electrode 25, the second lower buffer layer 37, the second active layer 36, the second upper buffer layer 38, the second upper electrode 26, and the common electrode 29 are stacked in this order on the sensor substrate 21.

In the detection device lA according to the modification, the stacking structure of the optical sensor PD is the same as that described above with reference to FIG. 9, and will not be described again. However, the first upper electrode 24 and the common electrode 29 of the optical sensor PD each have a light-transmitting property, and the first lower electrode 23 does not have a light- transmitting property. That is, the optical sensor PD is configured as a top-illuminated solar cell. The light Li emitted from the light sources 53 and 54 and transmitted through or reflected by the object to be detected Fg irradiates the first upper electrode 24 side of the optical sensor PD. That is, the light Li passes through the sealing film 90 and through the common electrode 29 and the first upper electrode 24 of the optical sensor PD, and irradiates the first active layer 31.

The second lower electrode 25 of the solar cell SC has a light-transmitting property, and the second upper electrode 26 does not have a light-transmitting property. That is, the solar cell SC is configured as a bottom- illuminated solar cell. The natural light L2 from outside irradiates the second lower electrode 25 side of the solar cell SC. That is, the natural light L2 passes through the sensor substrate 21 and through the second lower electrode 25 of the solar cell SC, and irradiates the second active layer 36.

Thus, the light Li transmitted through or reflected by the object to be detected Fg irradiates the optical sensor PD, and the natural light L2 is blocked by the first lower electrode 23. The natural light L2 irradiates the solar cell SC, and the light Li transmitted through or reflected by the object to be detected Fg is blocked by the second upper electrode 26. Thus, the detection device lA according to the modification can detect the light and generate the electricity satisfactorily using the optical sensor PD and the solar cell SC that are provided on the same sensor substrate 21.

When the optical sensor PD and the solar cell SC illustrated in FIG. 13 are accommodated in the housing 200 illustrated in FIG. 2, the lower surface of the sensor substrate 21 is placed facing the outer peripheral surfaces 201b and 202b of the housing 200, and the common electrode 29 and the sealing film 90 are placed facing the inner peripheral surfaces 201a and 202a of the housing 200.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiment and the modification thereof described above.

What is claimed is:

1. A detection device comprising:
a substrate;
an organic optical sensor in which a first lower electrode, a first lower buffer layer, a first active layer, a first upper buffer layer, a first upper electrode, and a common electrode are stacked in the order as listed, in a detection area of the substrate;
an organic photovoltaic device in which a second lower electrode, a second lower buffer layer, a second active layer, a second upper buffer layer, and the common electrode are stacked in the order as listed, in the detection area of the substrate; and
a sealing film that covers the organic optical sensor and the organic photovoltaic device, wherein the first lower electrode of the organic optical sensor and the common electrode of the organic photovoltaic device each have a light-transmitting property.

2. The detection device according to claim 1, wherein the sealing film is continuously provided across the organic optical sensor and the organic photovoltaic device, and the first lower electrode, the first lower buffer layer, the first active layer, the first upper buffer layer, and the first upper electrode of the organic optical sensor are provided so as to be separated from the second lower electrode, the second lower buffer layer, the second active layer, and the second upper buffer layer of the organic photovoltaic device.

3. The detection device according to claim 1, wherein the first lower buffer layer, the first active layer, and the first upper buffer layer of the organic optical sensor are formed of the same materials as those of the second lower buffer layer, the second active layer, and the second upper buffer layer of the organic photovoltaic device, respectively.

4. The detection device according to claim 1, comprising a light source configured to emit light to an object to be detected, wherein the light emitted from the light source and transmitted through or reflected by the object to be detected irradiates the first lower electrode side of the organic optical sensor.

5. The detection device according to claim 1, comprising a thin-film transistor (TFT) layer, an organic insulating film, and an inorganic insulating film stacked on the
substrate in the order as listed, wherein the organic optical sensor and the organic photovoltaic device are provided on the organic insulating film.

6. The detection device according to claim 1, comprising:
a detection circuit coupled to the organic optical sensor;
and a battery circuit coupled to the organic photovoltaic device.

7. The detection device according to claim 1, comprising a plurality of the organic optical sensors.

8. The detection device according to claim 1, comprising an annular housing, wherein
the housing comprises a first part having a light- transmitting inner peripheral surface and a non-light- transmitting outer peripheral surface and a second part having a light-transmitting outer peripheral surface and a non-light-transmitting inner peripheral surface,
each of the first and second parts includes at least one of the organic optical sensors and at least one of the organic photovoltaic devices,
in the first part, a total area of the at least one organic optical sensor is larger than a total area of the at least one organic photovoltaic device, and
in the second part, the total area of the at least one organic optical sensor is smaller than the total area of the at least one organic photovoltaic device.

9. The detection device according to claim 1, comprising an annular housing, wherein
the housing comprises a first part having a light-transmitting inner peripheral surface and a second part having a non-light-transmitting outer peripheral surface,
the organic optical sensor is located in the first part, and
the organic photovoltaic device is located in the second part.

10. A detection device comprising:
a substrate;
an organic optical sensor in which a first lower electrode, a first lower buffer layer, a first active layer, a first upper buffer layer, a first upper electrode, and a common electrode are stacked in the order as listed, in a detection area of the substrate; an organic photovoltaic device in which a second lower electrode, a second lower buffer layer, a second active layer, a second upper buffer layer, a second upper electrode, and the common electrode are stacked in the order as listed, in the detection area of the substrate; and
a sealing film that covers the organic optical sensor and the organic photovoltaic device, wherein
the first upper electrode and the common electrode of the organic optical sensor and the second lower electrode of the organic photovoltaic device each have a light- transmitting property, and
the second upper electrode of the organic photovoltaic device has no light-transmitting property.

11. The detection device according to claim 10, wherein the sealing film is continuously provided across the organic optical sensor and the organic photovoltaic device, and the first lower electrode, the first lower buffer layer, the first active layer, the first upper buffer layer, and the first upper electrode of the organic optical sensor are provided so as to be separated from the second lower electrode, the second lower buffer layer, the second active layer, and the second upper buffer layer of the organic photovoltaic device.

12. The detection device according to claim 10, wherein the first lower buffer layer, the first active layer, and the first upper buffer layer of the organic optical sensor are formed of the same materials as those of the second lower buffer layer, the second active layer, and the second upper buffer layer of the organic photovoltaic device, respectively.

13. The detection device according to claim 10, comprising a light source configured to emit light to an object to be detected, wherein the light emitted from the light source and transmitted through or reflected by the object to be detected irradiates the first upper electrode side of the organic optical sensor.

14. The detection device according to claim 10, comprising a thin-film transistor (TFT) layer, an organic insulating film, and an inorganic insulating film stacked on the substrate in the order as listed, wherein the organic optical sensor and the organic photovoltaic device are provided on the organic insulating film.

15. The detection device according to claim 10, comprising:
a detection circuit coupled to the organic optical sensor;
and a battery circuit coupled to the organic photovoltaic device.

16. The detection device according to claim 10, comprising a plurality of the organic optical sensors.

17. The detection device according to claim 10, comprising an annular housing, wherein
the housing comprises a first part having a light- transmitting inner peripheral surface and a non-light- transmitting outer peripheral surface and a second part having a light-transmitting outer peripheral surface and a non-light-transmitting inner peripheral surface,
each of the first and second parts includes at least one of the organic optical sensors and at least one of the organic photovoltaic devices,
in the first part, a total area of the at least one organic optical sensor is larger than a total area of the at least one organic photovoltaic device, and
in the second part, the total area of the at least one organic optical sensor is smaller than the total area of the at least one organic photovoltaic device.

18. The detection device according to claim 10, comprising an annular housing, wherein the housing comprises a first part having a light- transmitting inner peripheral surface and a second part having a non-light-transmitting outer peripheral surface, the organic optical sensor is located in the first part, and the organic photovoltaic device is located in the second part.
